(19) United States
(12) Patent Application Publication
(10) Pub. No.: US 2022/0081727 A1
(43) Pub. Date: Mar. 17, 2022

(54) BIOMARKER FOR PREDICTING RESPONSE TO ANTICANCER AGENT AND USE THEREOF

(71) Applicant: GENINUS INC., Seoul (KR)

(72) Inventors: Kyoung Mee KIM, Seoul (KR); Jee Yun LEE, Seoul (KR)

(21) Appl. No.: 17/423,929

(22) PCT Filed: Dec. 2, 2019
(86) PCT No.: PCT/KR2019/016866

§ 371 Date: Jul. 19, 2021

(30) Foreign Application Priority Data
Jan. 17, 2019 (KR) 10-2019-0006214
Nov. 28, 2019 (KR) 10-2019-0155534

(51) Int. Cl. C12Q 1/6886; G01N 33/574

(52) U.S. Cl. CPC C12Q 1/6886; G01N 33/57496; C12Q 2600/158; G01N 2333/912; G01N 2333/4703; G01N 2333/916; C12Q 2600/118

(57) ABSTRACT

The present disclosure describes a biomarker for predicting a response to an anticancer agent for immunotherapy, and more particularly, to a marker composition for predicting a response to a programmed cell death protein 1 (PD-1) inhibitor, pembrolizumab, which includes one or more genes selected from the group consisting of armadillo repeat-containing X-linked protein 1 (ARMCX1; NCBI accession number: NM_016608), serine/threonine-protein kinase D1 (PRKD1; NCBI accession number: NM_002742) and tyrosine kinase 2 (TYK2; NCBI accession number: NM_003331) or a protein encoded by the gene, and a composition, kit and information providing method for predicting a response to an anticancer agent. Since the gene marker according to the present disclosure utilizes a formalin-fixed paraffin-embedded tissue derived from a patient for analysis, separate sampling is not needed, and thus analysis is convenient.

### CROSS-REFERENCE TO RELATED APPLICATION

This application claims priority to and the benefit of Korean Patent Application No. 2019-0155534, filed on Nov. 28, 2019, the disclosure of which is incorporated herein by reference in its entirety.

### FIELD

The present disclosure describes a biomarker for predicting a response to an anticancer agent and a use thereof, and more particularly, to a marker composition for predicting a response to a programmed cell death protein 1 (PD-1) antagonist, pembrolizumab, which includes one or more genes selected from the group consisting of armadillo repeat-containing X-linked protein 1 (ARMCX1), serine/threonine-protein kinase D1 (PRKD1) and tyrosine kinase 2 (TYK2) or a protein(s) encoding the gene(s), and a composition, a kit and a method for predicting a response to the drug.

### BACKGROUND

Gastric cancer (GC) is one of the most common malignant tumors in the world, and is known to be the third leading cause of death from cancer. In addition, it has been reported that most GC patients are at an advanced stage and mostly have a very poor prognosis. In the case of metastatic GC, platinum-based combination chemotherapy is considered a standard therapy, but it is known that many patients are refractory to this therapy, and even in patients with a response, a response period is as short as several months. The reasons why GC has a low survival rate are that GC is a heterogeneous disease showing different aggressiveness and responsiveness to therapy, and clinical outcomes and prognosis for each patient are not consistent with reported data. Therefore, in the anticancer treatment for GC patients, if the most appropriate treatment is provided for GC patients having different characteristics by predicting the response and prognosis to a drug, it will be possible to avoid unnecessary treatment-related toxicity and ultimately increase a therapeutic effect.

According to such needs, recently, research on companion diagnostics, which means an approved diagnosis that can select appropriate target anticancer agent and treatment method based on the systemic analysis results of patient's personal factors, is actively progressing. Companion diagnostics may present clear clinical evidence for a prescription based on a doctor's diagnosis, present an appropriate treatment method to a patient to increase cancer treatment efficiency, and reduce the misuse of a targeted anticancer agent to contribute to the financial soundness of national health insurance. The current companion diagnostics market is growing in the fields of treatment of breast cancer, lung cancer, colorectal cancer, stomach cancer, melanoma, etc., and particularly, breast cancer and lung cancer are expected to drive the market growth. As pharmaceutical companies reduce new drug development costs and increase the demand for targeted therapies, the global market for companion diagnostics is expected to grow by 18% annually to reach 5.8 billion dollars in 2019.

Meanwhile, pembrolizumab approved by the FDA as an antibody against PD-1 is used in treatment of a late solid tumor patient with a microsatellite unstable (MSI)/DNA mismatch repair (dMMR)-deficient biomarker. Recently, as results of a nonrandomized, multicenter and multicohort basket test for pembrolizumab, which were conducted on 475 patients with one of 20 different PD-L1-positive advanced solid tumors, various objective response rates (ORRs) were exhibited across different cancer types. Biomarkers that can predict a response to anti-PD-1 therapy across various tumor types include a T-cell-inflammatory gene expression profile, PD-L1 expression, and/or a tumor mutational burden (TMB). However, larger-scale research is needed to more precisely assess clinical utility for such biomarkers in patient selection for anti-PD-1 therapy within individual cancer types.

In the case of GC, a single-arm, multicohort, pembrolizumab phase 2 trial (KEYNOTE-059) showed an ORR of 11.6%, and 2.3% of the patients showed a complete response (CR), and the responder group of the GC patients accounted for a lower proportion than patients with non-small cell lung cancer. The high proportion of the non-responder group and the emergence of resistance in patients who show initial responses pose very important challenges in the field of cancer immunotherapy. A recent study on the molecular characterization for metastatic GC patients treated with pembrolizumab showed a decrease in circulating tumor DNA levels at 6 weeks after treatment associated with MSI, Epstein-Barr virus (EBV)-positivity, PD-L1 combined positive score (CPS), TMB, and a response to the treatment. Although T-cell inflamed GEP provided a biomarker potentially relevant for prediction of the clinical efficacy of pembrolizumab therapy across a set of 20 diverse solid tumors and selection of patients who may benefit from PD-1 inhibition, it did not predict a response in patients with metastatic GC.

Predictive biomarkers for immunotherapy differ from the traditional biomarkers used for targeted therapies because of the complexity of the immune response and tumor biology. Recently, the immuno-predictive score (IMPRES) based on 45 immune checkpoint genes was developed to predict responses to ICB in patients with melanoma. Considering the differences in tumor biology and need for clinical-grade biomarkers to guide the choice of agents to maximize the likelihood of patient benefit, the inventors developed a GC-specific gene expression set for predicting a response to pembrolizumab.

### SUMMARY

The inventors performed a NanoString assay with RNA extracted from formalin-fixed paraffin-embedded tissue from gastric cancer (GC) patients treated with pembrolizumab to find a gene marker for predicting a response to an immunotherapeutic drug, pembrolizumab, which is one of the programmed cell death protein 1 (PD-1) inhibitors, and analyzed a gene differentially expressed according to responses of the GC patients, thereby finding a biomarker for predicting a response to an anticancer agent according to the present disclosure. Based on this, the present disclosure was completed.

Therefore, the present disclosure provides a marker composition for predicting a response to an anticancer agent, which includes one or more genes selected from the group consisting of armadillo repeat-containing X-linked protein 1 (ARMCX1), serine/threonine-protein kinase D1 (PRKD1) and tyrosine kinase 2 (TYK2) or a protein(s) encoding the gene(s).

The present disclosure provides a composition for predicting a response to an anticancer agent, which includes an agent that measures a level of an mRNA of one or more genes selected from the group consisting of ARMCX1, PRKD1 and TYK2 or a protein encoded by the gene, and a kit for predicting a response to an anticancer agent, which includes the composition.

In addition, the present disclosure provides an information providing method for predicting a response to an anticancer agent, which includes measuring a level of mRNA of one or more genes selected from the group consisting of ARMCX1, PRKD1 and TYK2, or a protein encoded by the gene.

However, technical problems to be solved in the present disclosure are not limited to the above-described problems, and other problems which are not described herein will be fully understood by those of ordinary skill in the art from the following descriptions.

To achieve the purpose of the present disclosure, the present disclosure provides a marker composition for predicting a response to an anticancer agent, which includes one or more genes selected from the group consisting of ARMCX1 (NCBI accession number: NM_016608), PRKD1 (NCBI accession number: NM_002742) and TYK2 (NCBI accession number: NM_003331) or a protein encoded by the gene.

In one embodiment of the present disclosure, the marker composition may further include ubiquitin carboxy-terminal hydrolase L1 (UCHL1; NCBI accession number: NM_004181) gene or a protein encoded by the gene.

The present disclosure also provides a composition for predicting a response to an anticancer agent, which includes an agent measuring a level of mRNA of one or more genes selected from the group consisting of ARMCX1 (NCBI accession number: NM_016608), PRKD1 (NCBI accession number: NM_002742) and TYK2 (NCBI accession number: NM_003331) or a protein encoded by the gene.

In one embodiment of the present disclosure, the composition may further include an agent that measures a level of mRNA of UCHL1 (NCBI accession number: NM_004181) gene or a protein encoded by the gene.

In addition, the present disclosure provides a kit for predicting a response to an anticancer agent, which includes the composition.

In one embodiment of the present disclosure, the anticancer agent may be a PD-1 antagonist.

In another embodiment of the present disclosure, the PD-1 antagonist may be pembrolizumab.

In still another embodiment of the present disclosure, the pembrolizumab may be used in treatment of one or more carcinomas selected from the group consisting of gastric cancer, lung cancer, skin cancer, head and neck cancer, Hodgkin's lymphoma, kidney cancer, and urothelial cell carcinoma.

In yet another embodiment of the present disclosure, the agent that measures an mRNA level of the gene may be a sense and antisense primer or probe, which complementarily binds to mRNA of the gene.

In yet another embodiment of the present disclosure, the agent that measures a protein level may be an antibody which specifically binds to a protein encoded by the gene.

In addition, the present disclosure provides an information providing method for predicting a response to an anticancer agent, which includes measuring a level of mRNA of one or more genes selected from the group consisting of ARMCX1 (NCBI accession number: NM_016608), PRKD1 (NCBI accession number: NM_002742) and TYK2 (NCBI accession number: NM_003331) or a protein encoded by the gene.

In one embodiment of the present disclosure, the information providing method may further include measuring a level of mRNA of UCHL1 (NCBI accession number: NM_004181) gene or a protein encoded by the gene.

In another embodiment of the present disclosure, the mRNA expression level may be measured by one or more types of methods selected from the group consisting of NanoString nCounter analysis, polymerization chain reaction (PCR), reverse transcription PCR (RT-PCR), real-time PCR, RNase protection assay (RPA), a microarray, and northern blotting.

In still another embodiment of the present disclosure, the protein expression level may be measured by one or more types of methods selected from the group consisting of western blotting, radioimmunoassay (RIA), radioimmunodiffusion, enzyme-linked immunosorbent assay (ELISA), immunoprecipitation, flow cytometry, immunofluorescence, Ouchterlony double immunodiffusion, complement fixation assay, and a protein chip.

In yet another embodiment of the present disclosure, the biological sample may be cancer patient-derived tissue.

In yet another embodiment of the present disclosure, the tissue may be paraffin-embedded tissue.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1 schematically illustrates the sequence of research for finding a gene marker for predicting a response to pembrolizumab according to the present disclosure;

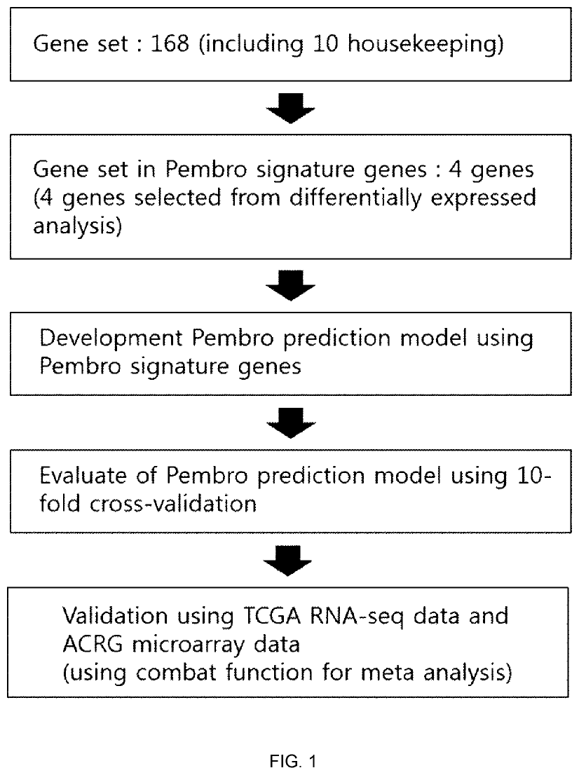

FIG. 2A is a volcano plot showing the result of analyzing a gene differentially expressed between the responder group and the non-responder group by response evaluation criteria in solid tumors (RECIST);

FIG. 2B shows the result of enrichment analysis of differentially expressed genes;

FIG. 2C shows a cutoff curve of IMAGiC scores for dividing patients into the two groups, and an AUC curve of IMAGiC scores for predicting a response to pembrolizumab;

FIG. 3A shows the result of analyzing the correlation between an IMAGiC model, a RECIST group, Epstein-Barr virus (EBV) and microsatellite instable (MSI);

FIG. 3B is a boxplot comparing IMAGiC scores between RECIST groups (CR: complete response, PR: partial response, SD: stable disease, PD: progressive disease);

FIG. 3C is a boxplot comparing IMAGiC scores between EBV subtypes (Positive: EBV positive, Negative: EBV negative);

FIG. 3D is a boxplot comparing IMAGiC scores between MSI subtypes (MSI: microsatellite instable, MSS: microsatellite stable);

FIG. 3E is a boxplot comparing IMAGiC scores between mutational loads (MLs; High ML, Mod ML, Low ML);

FIG. 3F is a boxplot comparing IMAGiC scores between programmed cell death protein 1 (PD-1) combined positive score (CPS) states;

FIG. 4A is a mutational spectrum in TCGA cohorts;

FIG. 4B is a boxplot comparing IMAGiC scores between MSI subtypes;

FIG. 4C is a boxplot comparing IMAGiC scores of hypermutated groups through tumor mutational loads;

FIG. 5A is a result of comparing differences in an overall survival rate and a disease-free survival rate between IMAGiC groups (responder, non-responder) in ACRG cohorts;

FIG. 5B is a boxplot comparing IMAGiC scores between ACRG subtypes (MSI, EMT, MSS/TP53+, MSS/TP53-);

FIG. 5C is a boxplot comparing IMAGiC scores between MSI subtypes;

FIG. 6A is the result of analyzing the correlation between an IMAGiC model, a RECIST group, EBV and MSI to validate the reproducibility of IMAGiC models using a qRT-PCR platform;

FIG. 6B is a boxplot comparing IMAGiC scores between RECIST groups;

FIG. 6C is a boxplot comparing IMAGiC scores between MSI subtypes; and

FIG. 6D is a boxplot comparing IMAGiC scores between EBV subtypes.

### DETAILED DESCRIPTION

As a result of attempting to conduct research to find biomarkers for predicting a response to the immunotherapeutic drug, pembrolizumab, the inventors found armadillo repeat-containing X-linked protein 1 (ARMCX1), serine/threonine-protein kinase D1 (PRKD1), tyrosine kinase 2 (TYK2) and ubiquitin carboxy-terminal hydrolase L1 (UCHL1) genes as biomarkers, and based on this, the present disclosure was completed.

Therefore, the present disclosure provides a marker composition for predicting a response to an anticancer agent, which includes one or more genes selected from the group consisting of armadillo repeat-containing X-linked protein 1 (ARMCX1; NCBI accession number: NM_016608), serine/threonine-protein kinase D1 (PRKD1; NCBI accession number: NM_002742) and tyrosine kinase 2 (TYK2; NCBI accession number: NM_003331) or a protein encoded by the gene.

In addition, the present disclosure provides a composition for predicting a response to an anticancer agent, which includes an agent measuring a level of mRNA of one or more genes selected from the group consisting of ARMCX1 (NCBI accession number: NM_016608), PRKD1 (NCBI accession number: NM_002742) and TYK2 (NCBI accession number: NM_003331) or a protein encoded by the gene, and a kit for predicting a response to an anticancer agent in cancer patients, which includes the composition.

In the present disclosure, the marker for predicting a response to an anticancer agent in cancer patients may further include a ubiquitin carboxy-terminal hydrolase L1 (UCHL1; NCBI accession number: NM_004181) gene or a protein encoded by the gene.

In the present disclosure, according to an exemplary embodiment, the above four types of gene markers were found, and specifically, their efficacy was validated for use in predicting a response to pembrolizumab treatment in gastric cancer (GC) patients.

In one embodiment of the present disclosure, gene expression profiling was analyzed from GC tissues derived from 21 GC patients with respect to a response to pembrolizumab, and four final genes, PRKD1, ARMCX1, TYK2 and UCHL1, were found through analysis of genes significantly differentially expressed between a responder group and a non-responder group based on response evaluation criteria in solid tumors (RECIST) (see Example 2).

In another embodiment of the present disclosure, to construct a model that can predict a response to pembrolizumab, IMAGiC models for predicting a response to pembrolizumab according to the present disclosure was constructed by performing linear regression analysis using mRNA expression levels of the selected four genes. Further, the sensitivity, specificity and accuracy of the models were analyzed, and based on IMAGiC scores for pembrolizumab and RECIST groups, the models were classified into a responder group and a non-responder group as IMAGiC groups. It was confirmed that the IMAGiC scores in the same cohorts have significant correlation with the RECIST groups (CR, PR, SD and PD; P=0.0057), an EBV-infected state (P=0.048) and tumor mutational load (ML; P=0.023) (see Example 3).

In still another embodiment of the present disclosure, the efficacy of the IMAGiC models according to the present disclosure was validated using RNA-sequencing data of The Cancer Genome Atlas (TCGA) and mRNA expression array results for Asia Cancer Research Group (ACRG) cohorts. More specifically, in TCGA cohorts, IMAGiC is significantly correlated with a TCGA molecular subtype (P=1.21E-04), MSI (P=0.01), EBV (P=0.09) and a TMB state (P=1.19E-05), confirming that IMAGiC models are associated with a recurrence-free survival rate, ACRG molecular subtype and MSI (see Example 4).

In yet another embodiment of the present disclosure, the reproducibility of IMAGiC models was assessed using a different technical method qRT-PCR, thereby confirming that the IMAGiC groups by qRT-PCR are also highly correlated with the RECIST group, EBV and MSI. Further, as a result of validating the result of IMAGiC qRT-PCR using a clinical cohort for nibolumab, the IMAGiC accuracy was 100% (see Example 5).

The term “anticancer agent” used herein is preferably an immunotherapeutic drug, which refers to a cancer therapy inducing an anticancer effect by stimulating an immune system. The anticancer agent used herein is more preferably a programmed cell death protein 1 (PD-1) antagonist, and more preferably, the PD-1 antagonist is pembrolizumab (trade name; Keytruda).

The term “antagonist” used herein refers to a material that acts antagonistically on the receptor binding of any bioactive material, but does not exhibit a physiological action via the receptor, and the PD-1 antagonist of the present disclosure, pembrolizumab, has a function of inhibiting the interaction with PD-L1 or PD-L2, which is a ligand of PD-1, expressed on the surface of cancer cells by binding to PD-1 that is expressed on the surface of an immune cell.

Among immunotherapies by the immunotherapeutic drug, passive immunotherapy is a treatment method of attacking cancer cells by injecting an immune response component made in vitro in a large amount, for example, an immune cell, antibody, or cytokine into a cancer patient, and active immunotherapy is a treatment method of attacking cancer cells by actively activating or producing an individual's antibody and immune cells. The present disclosure relates to a biomarker for predicting a response to pembrolizumab in such immunotherapy of a cancer patient and a use thereof.

In the present disclosure, the pembrolizumab may be used in treatment of one or more types of carcinomas selected from the group consisting of GC, lung cancer, skin cancer, head and neck cancer, Hodgkin's lymphoma, kidney cancer, and urothelial cell carcinoma, and more preferably GC, but the present disclosure is not limited thereto.

The “prediction of a response to an anticancer agent” used herein refers to a prediction of whether a patient favorably or unfavorably responds to an immunotherapeutic drug, a prediction of the risk of the resistance to an anticancer agent, and a prognosis of a patient after immunotherapy, that is, a prediction of recurrence, metastasis, survival or disease-free survival. The biomarker for predicting a treatment response according to the present disclosure may provide information to select the most appropriate immunotherapy method for a cancer patient, more specifically, a GC patient.

An agent for measuring an mRNA level of the marker gene for predicting a response to an anticancer agent may be a sense or antisense primer or probe, which complementarily binds to mRNA, but the present disclosure is not limited thereto.

The term “primer” refers to a short gene sequence that is the starting point of DNA synthesis, and an oligonucleotide synthesized to be used in diagnosis or DNA sequencing. The primers may be used after synthesis to a length of generally 15 to 30 bp, but may vary depending on the purpose of use, and may be modified through methylation or capping by a known method.

The term “probe” used herein refers to a nucleic acid that can be specifically bind to mRNA of several to hundreds of bp, which is manufactured through enzyme chemical separation and purification or synthesis. The presence or absence of mRNA may be confirmed by labeling a radioactive isotope, an enzyme or a fluorescent material, and may be designed or modified by a known method.

The agent for measuring a protein level may be an antibody specifically binding to a protein encoding a gene, but the present disclosure is not limited thereto.

The “antibody” used herein includes an immunoglobulin molecule having a response to an immunologically specific antigen, and includes both of a monoclonal antibody and a polyclonal antibody. In addition, the antibody includes all forms produced by genetic engineering, for example, a chimeric antibody (e.g., a humanized murine antibody) and a double-binding antibody (e.g., a bispecific antibody).

A kit for predicting a response to an anticancer agent according to the present disclosure may include a composition, solution or apparatus consisting of one or more types of different component compositions.

In another aspect of the present disclosure, the present disclosure provides an information providing method for predicting a response to an anticancer agent, which includes measuring a level of mRNA of one or more genes selected from the group consisting of ARMCX1 (NCBI accession number: NM_016608), PRKD1 (NCBI accession number: NM_002742) and TYK2 (NCBI accession number: NM_003331) or a protein encoded by the gene.

The mRNA expression level may be measured by one or more types of methods selected from the group consisting of NanoString nCounter analysis, polymerization chain reaction (PCR), reverse transcription PCR (RT-PCR), real-time PCR, RNase protection assay (RPA), a microarray, and northern blotting, but the present disclosure is not limited thereto.

The protein expression level may be measured by one or more types of methods selected from the group consisting of western blotting, radioimmunoassay (RIA), radioimmunodiffusion, enzyme-linked immunosorbent assay (ELISA), immunoprecipitation, flow cytometry, immunofluorescence, Ouchterlony double immunodiffusion, complement fixation assay, and a protein chip, but the present disclosure is not limited thereto.

The biological sample may be cancer patient-derived tissue, and more preferably, includes paraffin-embedded tissue which is fixed with a fixation liquid such a formalin, but the present disclosure is not limited thereto.

Hereinafter, to help in understanding the present disclosure, exemplary examples will be suggested. However, the following examples are merely provided to more easily understand the present disclosure, and not to limit the present disclosure.

### EXAMPLES

### Example 1. Experimental Preparation and Methods

1-1. Target GC Patient Selection

In this example, from 2013 to 2017, 61 patients with metastatic gastric cancer (mGC) were enrolled for a prospective single-site, phase 2 clinical trial to assess a clinical response to pembrolizumab. Among these patients, 21 patient cases were selected based on the availability of primary tumor tissue and clinical responses to pembrolizumab. As summarized in Table 1 below, the average age of the selected patients was 57 years (26 to 78 years), and the selected patients included four patients (19.05%) with microsatellite instable (MSI) GC, and two patients (9.52%) with EBV-positive GC, and clinical data, including demographic characteristics and treatment outcomes, were obtained by reviewing medical records.

TABLE 1

| | Patients (n = 21) |
|---|---|
| Age (years) | |
| | 57 (26-78) |
| Sex | |
| Male | 16 (76.19%) |
| Female | 5 (23.81%) |
| MSI | |
| MSI | 4 (19.05%) |
| MSS | 17 (80.95%) |
| EBV | |
| Positive | 2 (9.52%) |
| Negative | 19 (90.48%) |
| Response | |
| CR | 2 (9.52%) |
| PR | 4 (19.05%) |
| SD | 5 (23.81%) |
| PD | 10 (47.62%) |

1-2. RNA Extraction

To isolate total RNA from formalin-fixed paraffin-embedded tissue, each GC patient-derived tissue block was cut into 4-μm-thick sections. Subsequently, RNA was isolated using a RNeasy FFPE kit (Qiagen, Germany) according to the manufacturer's instructions. More specifically, the tissue section was deparaffinized, subjected to proteinase K treatment and on-column DNase digestion, followed by RNA extraction using RNase-free water. The total RNA sample isolated by the above-described method was stored at −80° C. until use, and an RNA concentration was measured using NanoDrop (Thermo Fisher Scientific, USA).

1-3. Gene Expression Profiling Using NanoString Analysis

For gene expression profiling, a NanoString analysis (NanoString Technologies, Inc, Seattle, USA) was performed using the RNA isolated from the GC tissue sample according to Example 1-2. For the analysis, the inventors formed probes for 168 genes related to mesenchymal signature and host immune response genes identified by comprehensive analysis of tumor immunity. For controls, 11 housekeeping genes and 14 technical internal control genes were added. Subsequently, NanoString analysis was performed according to the standard protocol “Setting up 12 nCounter Assays (MAN-00003-03, 2008-2013)”. A hybridization reaction was induced by incubation for 18 hours, data was analyzed using the nSolver software (NanoString Technologies). Data was normalized using housekeeping genes and internal control genes, and converted to a log₁₀ scale in nSolver software (version 4.0).

1-4. Differentially Expressed Gene Analysis According to Response to Pembrolizumab

To find a gene signature that can predict a response to pembrolizumab, differentially expressed gene (DEG) analysis was performed using nSolver software. To identify a DEG by comparing a group having a response to pembrolizumab and a group with no response, P<0.01 was selected as a cutoff value.

1-5. Development of Pembrolizumab Response Prediction Model and Validation in TCGA and ACRG Cohorts

To construct a prediction model for pembrolizumab, mRNA expression levels of genes with significantly different expression patterns and the PD-L1 CPS of the GC tissues were analyzed using a linear regression model. The prediction model was evaluated by calculating sensitivity and specificity with the area under the curve (AUC) of an ROC curve. Cutoff values for dividing mGC patients into a responder group and a non-responder group were defined by the accuracy function in an AUC package.

Further, to evaluate the prediction model according to the present disclosure, cross validation was performed 10 times, and the root mean squared error (RMSE) was calculated for each validation result. In addition, as MSI, EBV-positive and TMB are known to be closely related to responses to immunotherapy, the result of the present disclosure was validated using RNA sequencing data of TCGA and microarray data of ACRG. The ComBat function was used for adjusting gene expression data using the sva package since the validation data and test data have different platforms. All statistical analyses and visualized plots were performed in the R program (R version 3.4.4). The total mutational rate was calculated with the number of somatic non-synonymous single nucleotide variant (SNV) mutations per megabase, and the threshold for a high mutational load (ML) was set as the upper tertile.

1-6. Validation Using Quantitative Real-Time PCR

To evaluate reproducibility of IMAGiC models according to the present disclosure with different technical platforms, a condition in which quantitative real-time PCR was performed using a 7900HT sequence detection system (Applied Biosystems, Foster City, Calif., USA) in a 384-well plate with a final volume of 10 μL per reaction of a reaction solution containing 5 μL of 2X Taqman PreAmp Master Mix, 4 μL of a cDNA sample, and 1 μL of a primer/probe was set. PCR amplification was performed under the following conditions, and the same sample was amplified in three independent wells: 2 minutes at 50° C. and 10 minutes at 94° C., followed by 40 cycles of 15 seconds at 95° C. and 60 seconds at 60° C.

1-7. Immunohistochemistry for PD-L1

Immunohistochemistry (IHC) was performed using each representative section of formalin-fixed paraffin-embedded tissue (FFPE) samples. PD-L1 staining was conducted using an FDA-approved monoclonal mouse antibody, PD-L1 22C3 pharmDx (Dako, Carpinteria, Calif.). In addition, the results of PD-L1-stained IHC slides were interpreted by an experienced pathologist (KMK): CPS was calculated by summing the number of PD-L1-stained cells (tumor cells, lymphocytes and macrophages), dividing the result by the total number of viable tumor cells, and multiplying by 100, and assessed according to the PD-L1 IHC 22C3 pharmDx instructions for use (https://www.agilent.com/cs/library/usermanuals/public/29219_pd-11-ihc-22C3-pharmdx-gastric-interpretation-manual_us.pdf). PD-L1 IHC was interpreted as positive when the score was 1 or more, and negative when the score was less than 1.

### Example 2. Identification of Differentially Expressed Gene in Response to Pembrolizumab

Gene expression profiling was analyzed from GC tissues derived from 21 GC patients related to responses to pembrolizumab. Based on response evaluation criteria in solid tumors (RECIST), according to the response to pembrolizumab, the tissues were divided into four groups, in which a complete response (CR) was classified as four cases, a partial response (PR) was classified as four cases, stable disease (SD) was classified as 5 cases, and progressive disease (PD) was classified as 10 cases.

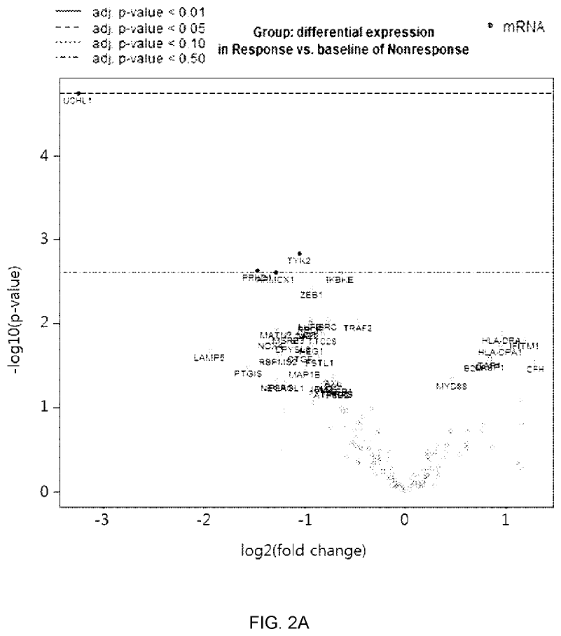

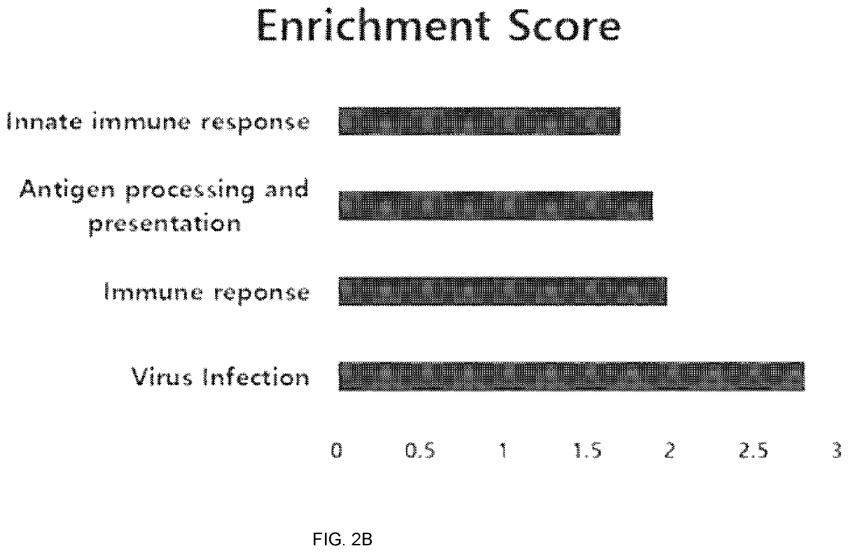

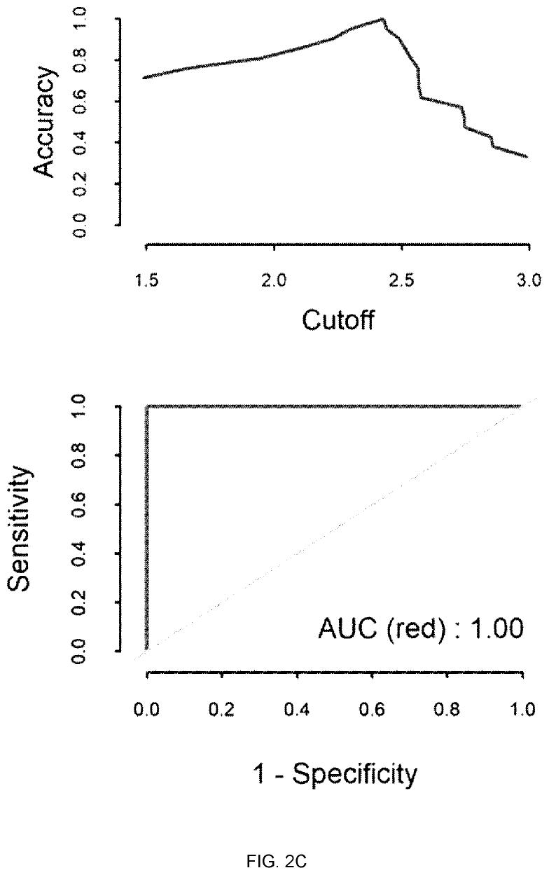

To screen signature genes for prediction of a response to pembrolizumab, DEG analyses were performed using nSolver software as shown in FIG. 2A. Afterward, enrichment analysis was performed using all DEGs (P<0.05). As a result, as shown in FIG. 2B, all DEGs were most significantly associated with EBV infection and immune responses as well as antigen processing and presentation and innate immune responses, indicating that the most important factors in predicting a response to pembrolizumab are associated with immune responses.

Therefore, finally, from the DEG analysis result of FIG. 2A, 4 genes significantly differentially expressed, such as UCHL1, PRKD1, ARMCX1 and TYK2, between a responder group and a non-responder group with respect to pembrolizumab treatment were selected (P<0.01).

### Example 3. Construction of IMAGiC Model for Predicting Response to Pembrolizumab

To construct a model that can predict a response to pembrolizumab, linear regression analysis was performed using, mRNA expression levels of 4 genes selected from DEG. In addition, since PD-L1 expression is an important biomarker for pembrolizumab responsiveness, PD-L1 CPS was also used for IMAGiC. Further, to evaluate the performance of the constructed IMAGiC model according to the above-described method, the model was validated in the same patient cohort. As a result, as shown in FIG. 2C, all IMAGiC groups matched perfectly in responses to pembrolizumab. The sensitivity, specificity and accuracy of the IMAGiC model were calculated using an AUC method, and all of them were confirmed to be 100%. Further, to reduce the bias of the selected data and improve the performance of the IMAGiC model, 10-fold cross validation was performed. All 21 samples were used to construct an IMAGiC model, and divided into 10 groups for each cross-validation step; nine of the 10 groups were used for testing, and the other group was used for validation. The average RMSE was 1.751.

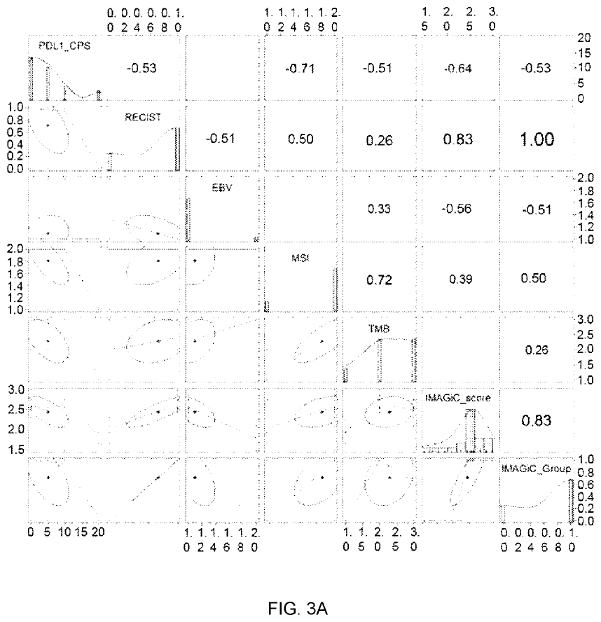

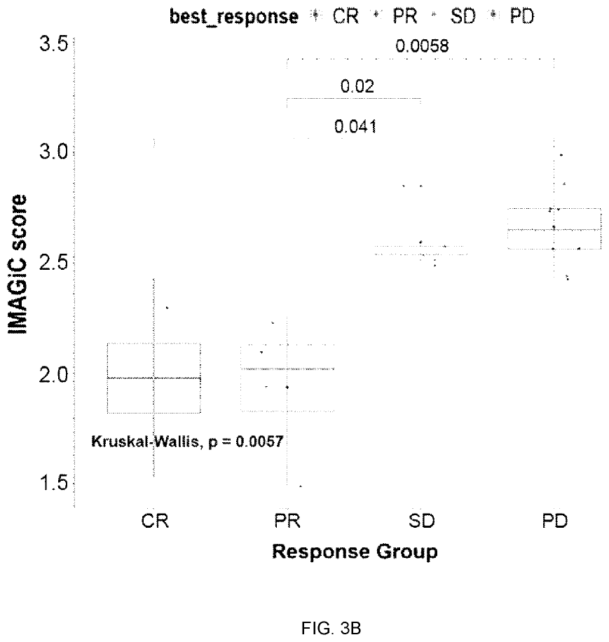

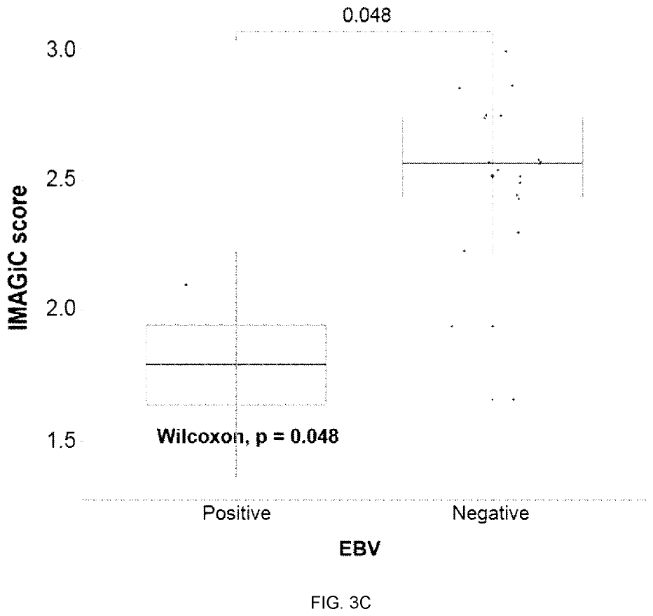

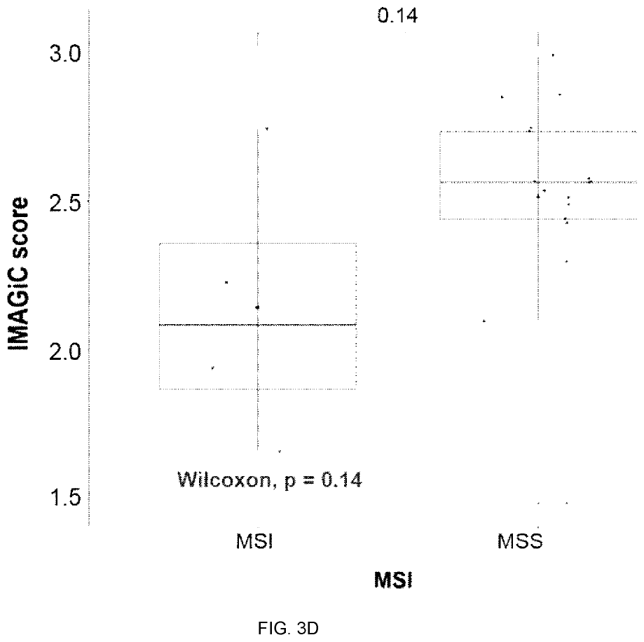

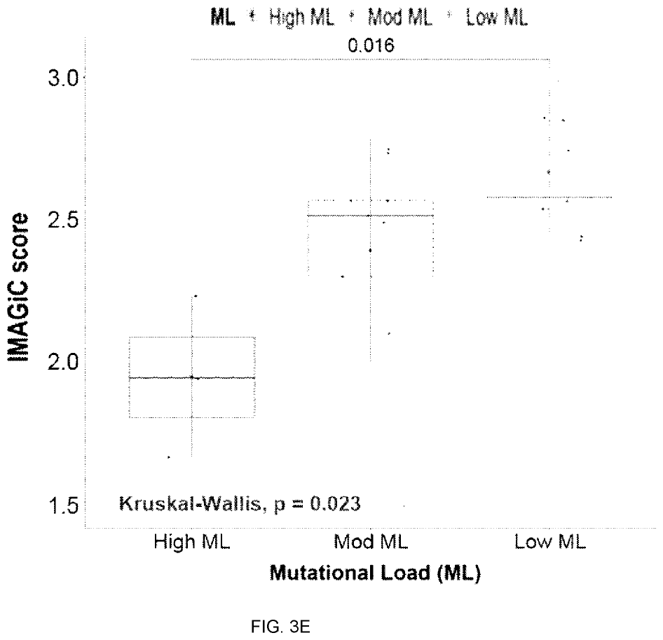

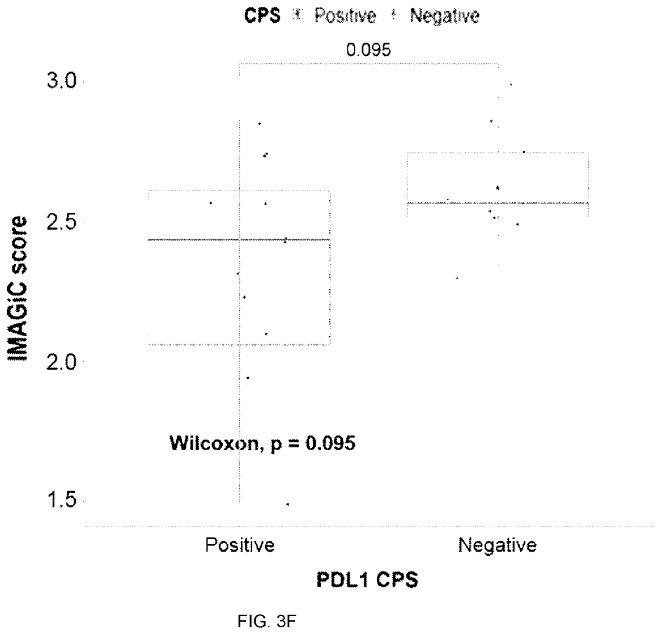

The IMAGiC group was developed based on the IMAGiC score and RECIST groups for pembrolizumab, and the cut off value of the IMAGiC score was set as 2.5069 using the accuracy function of the AUC package. Finally, mGC patients were divided into a responder group and a non-responder group based on IMAGiC. As shown in FIG. 3A, the IMAGiC score was shown to have a correlation with PD-L1 CPS (r²=0.64), a RECIST group (r²=0.83) and an EBV-infected state (r²=0.56). In addition, as shown in FIGS. 3B to 3F, the IMAGiC score was confirmed to have a significant correlation with RECIST groups (CR, PR, SD and PD; P=0.0057), EBV-infected state (P=0.048) and tumor mutational load (ML)(P=0.023). However, the IMAGiC score did not show an association with MSI subtype (P=0.14) and PD-L1 CPS (P=0.095).

### Example 4. Analysis of IMAGiC in TCGA and ACRG Cohorts

As it has been reported that conventional PD-1 blockade is effective against MSI and EBV-positive tumors as well as a tumor with high ML, the inventors applied the IMAGiC prediction model for TCGA RNA-sequencing data (n=260) and ACRG mRNA expression array results (n=300) for validation.

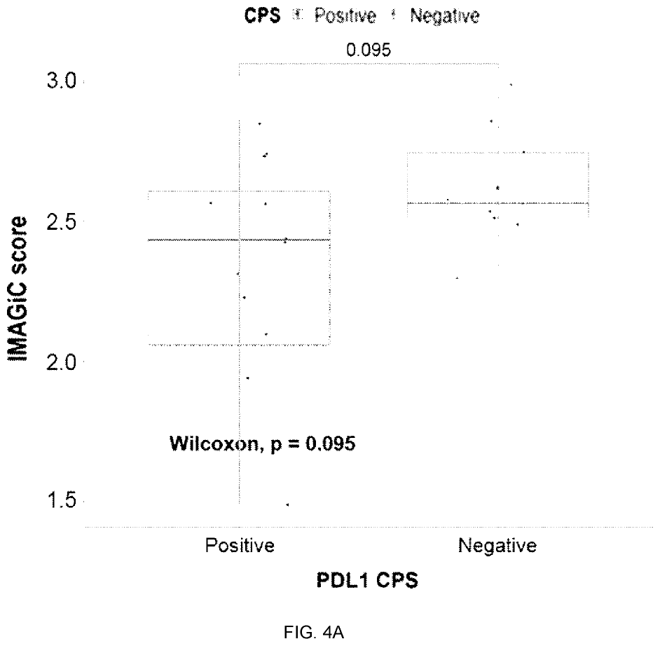

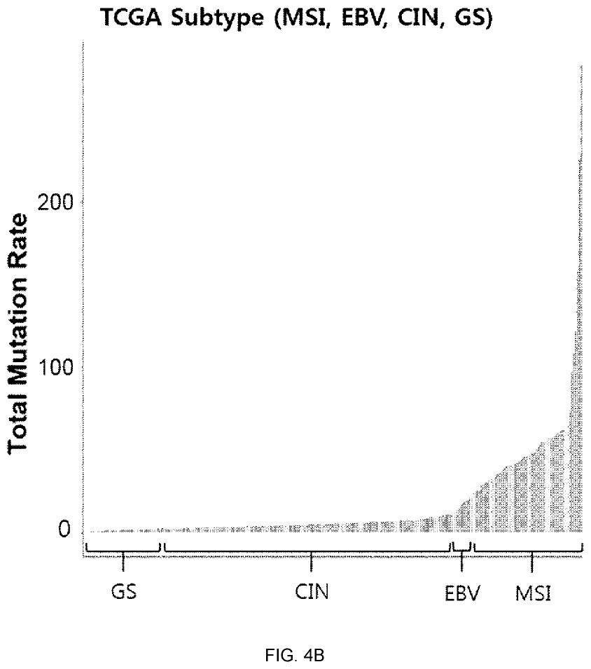

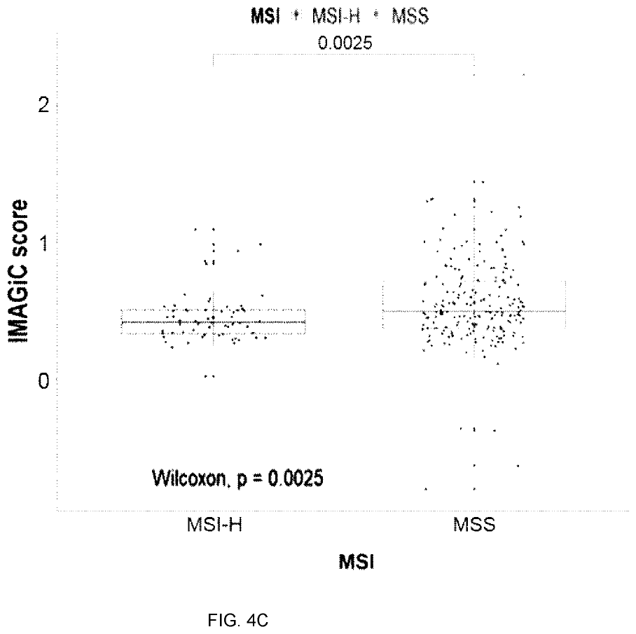

First, as a result of analyzing GC subtypes in the TCGA group, as shown in FIG. 4A, there were 59 cases with MSI GC (22.7%), 24 cases of EBV-positive GC (9.2%), and 61 cases of TMB-high subtype GC (23.5%), and the total mutation rate was distinct in four molecular subtypes. Further, as a result of analyzing the correlation between subtype GCs included in the IMAGiC and TCGA cohorts, as shown in Table 2 below, IMAGiC significantly correlated with the TCGA molecular subtype (P=1.21E-04), MSI (P=0.01), EBV(P=0.09) and the TMB state (P=1.19E-05). That is, many patients with MSI or EBV-positive GC were classified as a responder group by IMAGiC. In addition, as shown in FIGS. 4B and 4C, it was confirmed that the IMAGiC scores in GC associated with MSI and TMB were significantly higher than those in microsatellite-stable (MSS) and non-TMB groups.

TABLE 2

| | Responders | Non-responders | |
|---|---|---|---|
| IMAGiC Group | (n = 125) | (n = 135) | p-value |
| Sex | | | 0.7102 |
| Male | 74 (46.84%) | 84 (53.16%) | |
| Female | 51 (50.00%) | 51 (50.00%) | |
| TCGA subtype | | | 1.21E−04 |
| EBV | 16 (66.67%) | 8 (33.33%) | |
| MSI | 37 (62.71%) | 22 (37.29%) | |
| CIN | 60 (47.62%) | 66 (52.38%) | |
| GS | 12 (23.53%) | 39 (76.47%) | |
| MSI | | | 0.0159 |
| MSI-H | 37 (62.71%) | 22 (37.29%) | |
| MSS | 88 (43.78%) | 113 (56.22%) | |
| EBV | | | 0.0894 |
| Positive | 16 (66.67%) | 8 (33.33%) | |
| Negative | 109 (46.19%) | 127 (53.81%) | |
| Total mutation rate* | 21.2401 (±34.4094) | 12.6771 (±25.5755) | 1.19E−05 |

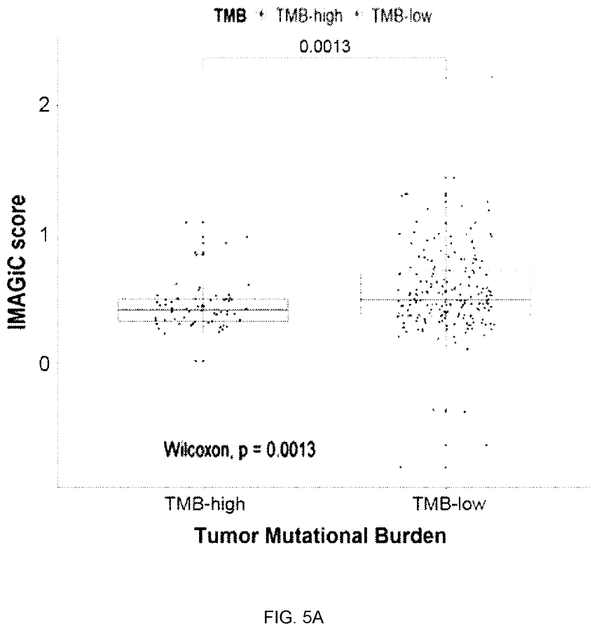

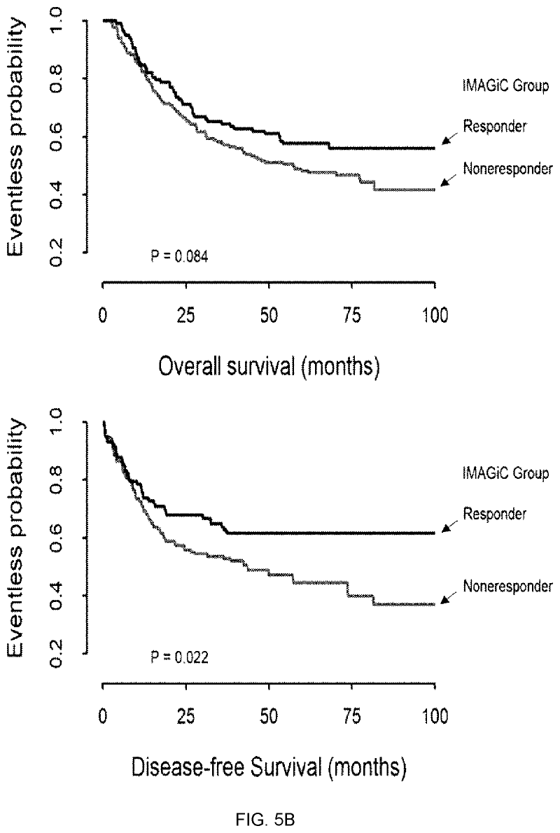

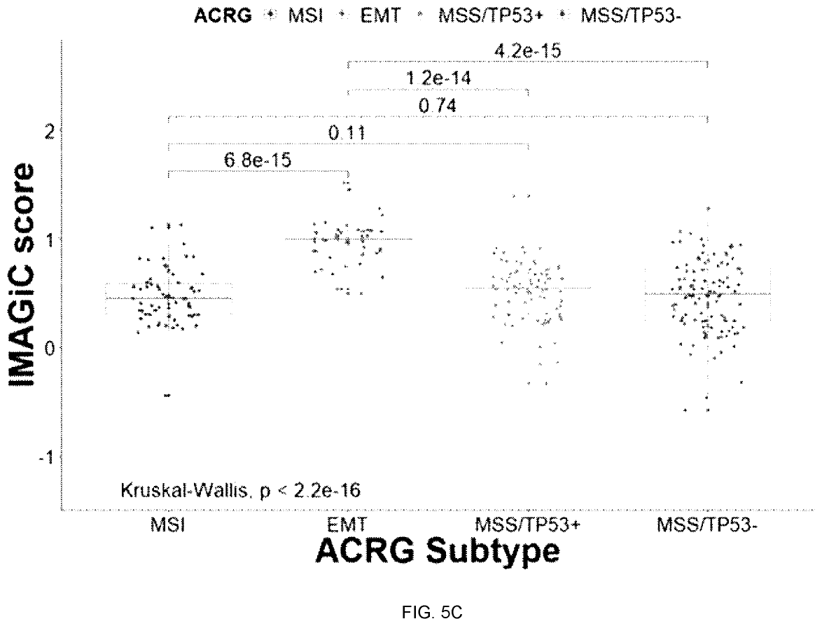

Meanwhile, in the ACRG cohort, as shown in Table 3 below, there were 68 cases of MSI GC (22.7%) and 18 cases of EBV-positive GC (6.0%). In addition, the differences in the overall survival rate and disease-free survival rate between IMAGiC groups, such as a responder group and a non-responder group were compared, and it was confirmed that, as a result of comparing the IMAGiC scores in the ACRG molecular subtypes (MSI, EMT, MSS/TP53+, and MSS/TP53-) and the MSI subtype (MSI-H, MSS) groups, as shown in FIGS. 5A to 5C, the IMAGiC model was associated with the disease-free survival rate, the ACRG molecular subtypes (P=1.96E-08) and MSI (P=0.006).

TABLE 3

| | Responders | Non-responders | |
|---|---|---|---|
| IMAGiC Group | (n = 118) | (n = 182) | p-value |
| Sex | | | 0.0396 |
| Male | 87 (43.72%) | 112 (56.28%) | |
| Female | 31 (30.69%) | 70 (69.31%) | |
| ACRG subtype | | | 1.96E−08 |
| MSI | 37 (54.41%) | 31 (45.59%) | |
| EMT | 0 (0.00%) | 46 (100.00%) | |
| MSS/TP53+ | 31 (39.24%) | 48 (60.76%) | |
| MSS/TP53− | 50 (46.73%) | 57 (53.27%) | |
| MSI | | | 0.0059 |
| MSI-H | 37 (54.41%) | 31 (45.59%) | |
| MSS | 81 (34.91%) | 151 (65.09%) | |
| EBV | | | 0.1116 |
| Positive | 3 (16.67%) | 15 (83.33%) | |
| Negative | 106 (41.25%) | 151 (58.75%) | |
| NA | 9 (36.00%) | 16 (64.00%) | |

### Example 5. Reproduction and Validation of IMAGiC Using qRT-PCR

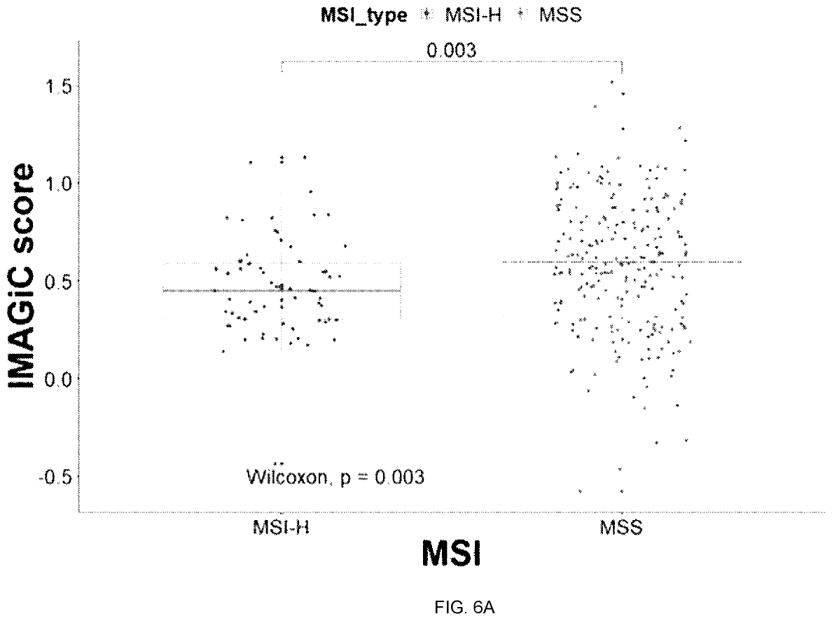

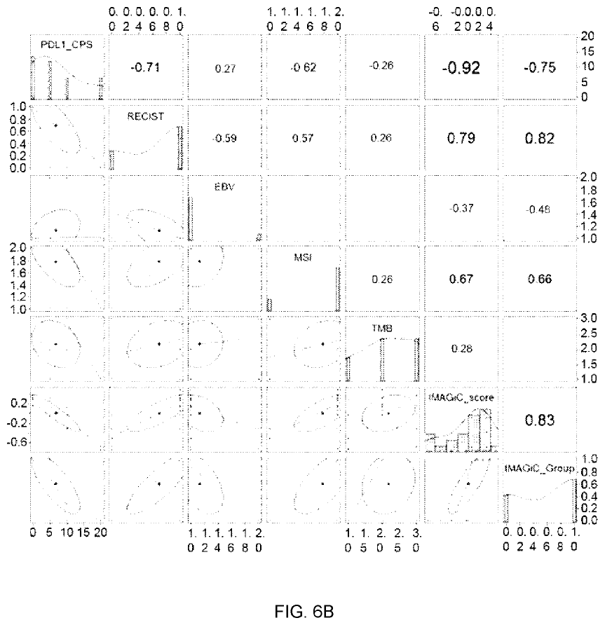

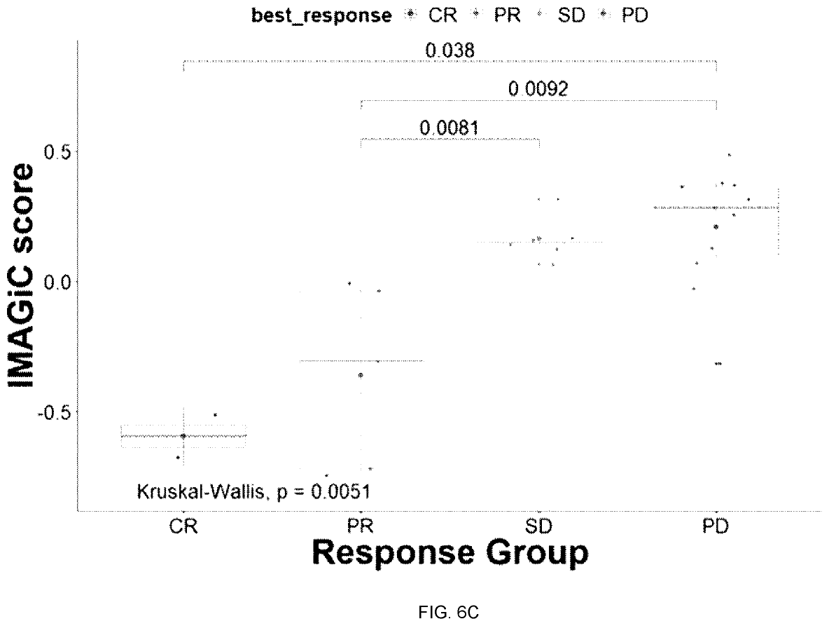

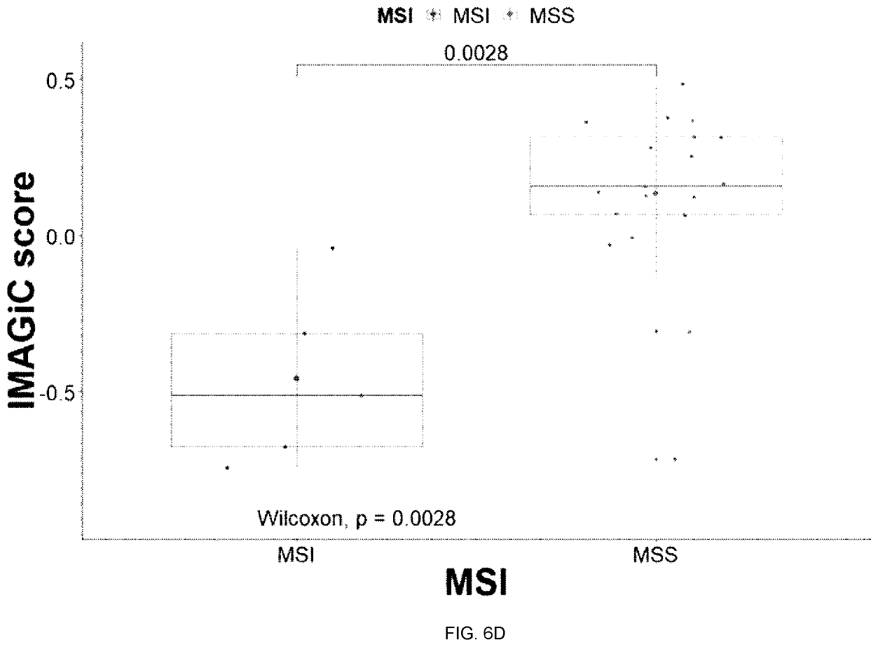

For use in clinical practice, the inventors were attempted to evaluate the reproducibility of the IMAGiC model with different technical methods. To this end, qRT-PCR was performed with mRNA from 24 patients in the same cohort. As a result, as shown in FIGS. 6A to 6D, the IMAGiC group by qRT-PCR was highly associated with the RECIST group (r²=0.82), EBV (r²=0.48) and MSI (r²=0.66). The accuracy for the reproducibility of IMAGiC was 87.5% (positive predictive value, 87.5%; negative predictive value, 12.5%).

To validate the results of IMAGiC qRT-PCR in different patient groups with mGC, the inventors used 17 patient samples obtained from an ongoing trial with nivolumab (Opdivo, Bristol-Myers Squibb Company Inc.). As shown in Table 4 below, all cases were EBV-positive, and most cases (94.1%) were MSS and PD-L1 CPS-negative. Consistent with recent trial and research results in which MSI, EBV-positive and positive PD-L1 CPS are associated with responses to pembrolizumab, most of the cases were classified as a non-responder group of the IMAGiC groups based on the RECIST groups. In addition, to investigate whether the IMAGiC qRT-PCR model has the same accuracy as that of the original model, the accuracy of IMAGiC was calculated using the nivolumab cohort, and as a result, the accuracy was 100% (positive predictive value, 100%; negative predictive value, 0%).

TABLE 4

| | TMB (Tumor | | | PD-L1 | IMAGiC | |
|---|---|---|---|---|---|---|
| Sample | Mutation Burden) | MSI | EBV | CPS | score | IMAGiC group |
| 1 | 9.27 | MSS | Negative | 0 | 0.9722 | Non-responder |
| 2 | 10.93 | MSS | Negative | 0 | 0.3100 | Non-responder |
| 3 | 3.37 | MSS | Negative | 0 | 1.3076 | Non-responder |
| 4 | 1.68 | MSS | Negative | 0 | 0.3926 | Non-responder |
| 5 | 4.2 | MSS | Negative | 0 | 0.3314 | Non-responder |
| 6 | 3.35 | MSS | Negative | 0 | 0.3534 | Non-responder |
| 7 | 0 | MSS | Negative | 0 | 0.3726 | Non-responder |
| 8 | 3.38 | MSS | Negative | 0 | 0.2417 | Non-responder |
| 9 | 6.75 | MSS | Negative | 0 | 0.2220 | Non-responder |
| 10 | 5.04 | MSS | Negative | 0 | 0.4416 | Non-responder |
| 11 | 2.54 | MSS | Negative | 0 | 1.6643 | Non-responder |
| 12 | 2.52 | MSS | Negative | 0 | 0.2412 | Non-responder |
| 13 | 5.05 | MSS | Negative | 0 | 1.0175 | Non-responder |
| 14 | 5.9 | MSS | Negative | 0 | 0.3310 | Non-responder |
| 15 | 2.53 | MSS | Negative | 0 | 0.7392 | Non-responder |
| 16 | 10.11 | MSI-H | Negative | 1 | 0.3268 | Non-responder |
| 17 | 8.43 | MSS | Negative | 0 | 0.8758 | Non-responder |

It should be understood by those of ordinary skill in the art that the above description of the present disclosure is exemplary, and the exemplary embodiments disclosed herein can be easily modified into other specific forms without departing from the technical spirit or essential features of the present disclosure. Therefore, the exemplary embodiments described above should be interpreted as illustrative and not limited in any aspect.

A gene marker found in the present disclosure may be used to predict a response to an immunotherapeutic drug, pembrolizumab, and thus may be used to select the optimal therapy for individual cancer patients, resulting in an increase in therapeutic effect and minimization of side effects. Therefore, the marker may be effectively used in clinical field of companion diagnostics.

The inventors found a gene set for predicting a response to an anticancer agent using a tissue sample derived from a gastric cancer (GC) patient treated with the immunotherapeutic drug, pembrolizumab, and validated the efficiency of the markers using microarray data of Asian Cancer Research Group (ACRG) and the results from RNA sequencing data of The Cancer Genome Atlas (TCGA) cohorts. As a result, the gene markers according to the present disclosure are convenient for analysis since analysis is performed using patient-derived formalin-fixed paraffin-embedded tissues without separate sampling, and provide information for selecting an optimal treatment method by predicting a response to the immunotherapy in advance, and therefore are expected to be effectively used in the clinical companion diagnostics field.

It will be apparent to those skilled in the art that various modifications can be made to the above-described exemplary embodiments of the present disclosure without departing from the spirit or scope of the invention. Thus, it is intended that the present disclosure covers all such modifications provided they come within the scope of the appended claims and their equivalents.

1. A method of predicting a response to an anticancer agent, comprising:
measuring a level of mRNA of one or more genes selected from the group consisting of armadillo repeat-containing X-linked protein 1 (ARMCX1; NCBI accession number: NM_016608), serine/threonine-protein kinase D1 (PRKD1; NCBI accession number: NM_002742) and tyrosine kinase 2 (TYK2; NCBI accession number: NM_003331) or a protein encoded by the gene.

2. The method according to claim 1, further comprising:
a ubiquitin carboxy-terminal hydrolase L1 (UCHL1; NCBI accession number: NM_004181) gene or a protein encoded by the gene.

3. The method according to claim 1, wherein the anticancer agent is a programmed cell death protein 1 (PD-1) antagonist.

4. The method according to claim 3, wherein the PD-1 antagonist is pembrolizumab.

5. The method according to claim 4, wherein the pembrolizumab is used in treatment of one or more carcinomas selected from the group consisting of gastric cancer, lung cancer, skin cancer, head and neck cancer, Hodgkin's lymphoma, kidney cancer, and urothelial cell carcinoma.

6.-10. (canceled)

11. The method of claim 1, wherein the agent for measuring an mRNA level of the gene is a sense or antisense primer or probe, which complementarily binds to mRNA of the gene.

12. The method of claim 1, wherein the agent for measuring a protein level is an antibody specifically binding to the protein encoded by the gene.

13. A kit for predicting a response to an anticancer agent, comprising an agent for measuring an one or more genes selected from the group consisting of armadillo repeat-containing X-linked protein 1 (ARMCX1; NCBI accession number: NM_016608), serine/threonine-protein kinase D1 (PRKD1; NCBI accession number: NM_002742) and tyrosine kinase 2 (TYK2; NCBI accession number: NM_003331) gene, or an agent for measuring a level of a protein encoded by the gene.

14.-15. (canceled)

16. The method of claim 1, wherein the mRNA level is measured by one or more methods selected from the group consisting of NanoString nCounter analysis, polymerization chain reaction (PCR), reverse transcription PCR (RT-PCR), real-time PCR, RNase protection assay (RPA), a microarray, and northern blotting.

17. The method of claim 1, wherein the protein level is measured by one or more methods selected from the group consisting of western blotting, radioimmunoassay (RIA), radioimmunodiffusion, enzyme-linked immunosorbent assay (ELISA), immunoprecipitation, flow cytometry, immunofluorescence, Ouchterlony double immunodiffusion, complement fixation assay, and a protein chip.

18. The method of claim 1, wherein the biological sample is cancer patient-derived tissue.

19. The method of claim 11, wherein the tissue is paraffin-embedded tissue.
